(19) United States
(12) Patent Application Publication
(10) Pub. No.: US 2015/0305620 A1
(43) Pub. Date: Oct. 29, 2015

(54) Self-Contained, Wireless Patient Monitoring Pad

(71) Applicant: Rondish Company Ltd., Kwai Chung (CN)

(72) Inventors: Steven Alfred Williams, Kwai Chung (CN); Edwin Lau, Kwai Chung (CN)

(21) Appl. No.: 14/696,911

(22) Filed: Apr. 27, 2015

Related U.S. Application Data
(60) Provisional application No. 61/984,612, filed on Apr. 25, 2014.

(51) Int. Cl. A61B 5/00; A61B 5/11

(52) U.S. Cl. CPC A61B 5/0022; A61B 5/1117; A61B 5/74; A61B 5/6892

(57) ABSTRACT

A self-contained, wireless patient monitoring pad has wireless communication components, such as a transceiver, and a power source, such as a battery, which are built in during manufacture of the self-contained, wireless patient monitoring pad. The self-contained, wireless patient monitoring pad communicates with a patient monitoring system that can include a central monitoring point. A user activates the power source in the self-contained, wireless patient monitoring pad prior to putting the monitoring pad into service or use, so that the self-contained, wireless patient monitoring pad can be stored indefinitely without dissipating the energy stored within built-in the power supply.

### REFERENCE TO PRIORITY DOCUMENT

The present application claims priority to U.S. Provisional Patent Application Ser. No. 61/984,612 entitled “SELF-CONTAINED, WIRELESS PATIENT MONITORING PAD” and filed on Apr. 25, 2014. Priority to the aforementioned filing date is claimed and the provisional application is incorporated by reference in its entirety.

### BACKGROUND

The present disclosure relates to systems, methods, and apparatuses for monitoring the status of a patient using self-contained, wireless patient monitoring pad that communicates with a patient monitoring system. The self-contained, wireless patient monitoring pad and the patient monitoring system communicate through transceivers within each of the wireless patient monitoring pad and the patient monitoring system. The self-contained, wireless patient monitoring pad includes a power supply and one or means to selectively activate the patient monitoring pad.

Patient monitoring systems are often based upon a wired connection between a sensor system, such as a bed-pad or floor mat, and a monitoring apparatus located either next to the patient or at times away from the patient, such as at a nurses' station. When multiple types of sensors and sensor systems are used to monitor a patient for unwanted movement, such as falls, wired connections can become burdensome and a safety hazard. The wires attached to a patient monitoring pad can include lines to send and receive signals, to provide power, to charge a battery, or a combination thereof.

Patient monitoring pads can be complex, with numerous wired connections and lines for communication and power needs, so wireless communication and power can simplify the monitoring system. Also, it can be desirable to have a patient monitoring pad that is disposable, such that the patient monitoring pad is good for a finite period of time or such that one patient monitoring pad can be used for the duration of a patient's hospital stay.

### SUMMARY

Disclosed is a self-contained, wireless patient monitoring pad that communicates with a patient monitoring system wirelessly via transceivers and has a built-in power supply. The self-contained, wireless patient monitoring pad is easy to implement and can be stored indefinitely.

Other features and advantages should be apparent from the following description of various embodiments, which illustrate, by way of example, the principles of the disclosure.

### BRIEF DESCRIPTION OF THE DRAWINGS

FIGS. 1A-1D are views of an exemplary self-contained, wireless patient monitoring pad;

FIG. 2 is a flow diagram showing potential flows of data between an exemplary self-contained, wireless patient monitoring pad and a monitoring system; and

FIG. 3 is a diagram showing an exemplary method of using a self-contained, wireless patient monitoring pad.

### DETAILED DESCRIPTION

A self-contained, wireless patient monitoring pad for use in a patient monitoring system is described herein. The self-contained, wireless patient monitoring pad and the patient monitoring system communicate wirelessly via transceivers. The self-contained, wireless patient monitoring pad has an internal transceiver and mobile power source, such as a battery.

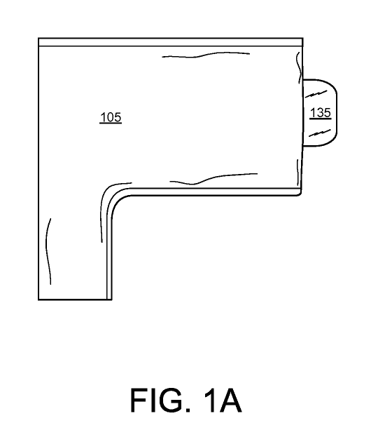

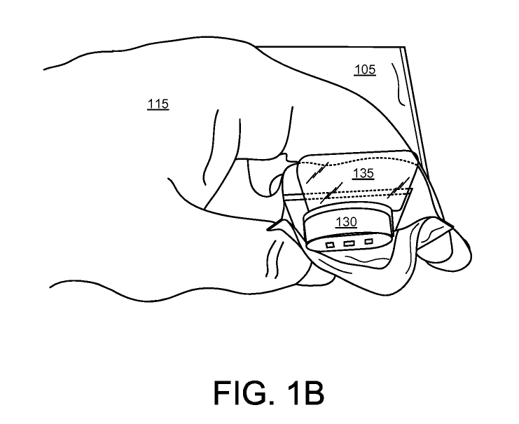

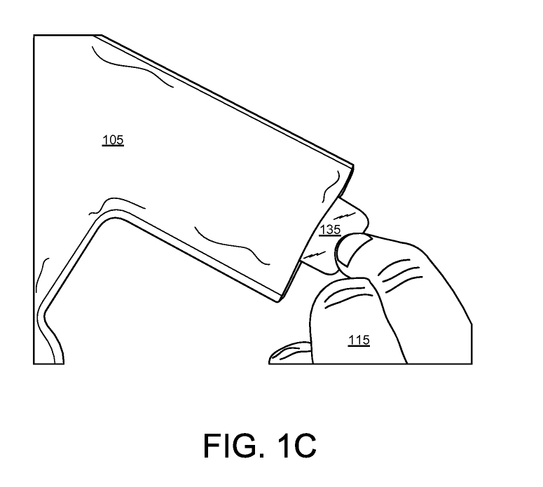

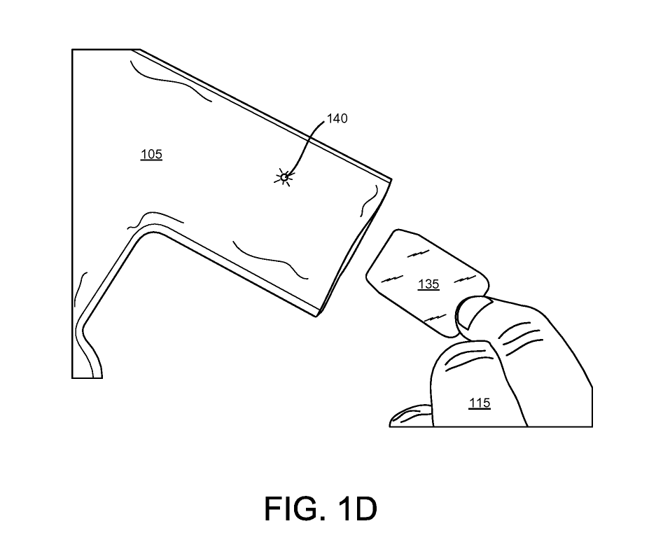

FIGS. 1A-1D are views of an exemplary self-contained, wireless patient monitoring pad 105 which may be a flexible container or case that defines an inner cavity that can contain one or more components. The exemplary self-contained, wireless patient monitoring pad 105 includes one or more circuits which include at least one printed circuit board (PCB), a transceiver, and a power supply 130 with a pull tab 135. Exemplary circuits are described in further detail in U.S. Patent Application Publication US 2010-0163315, and in U.S. patent application Ser. No. 14/075,133, filed Nov. 8, 2013 titled “Bed Monitoring Pad,” the disclosures of which are hereby incorporated by reference herein.

The power supply 130 and wireless communications components (not visible) are built into the patient monitoring pad 105 during the manufacturing stage. In some implementations, the power supply 130 is a battery with a finite lifetime, intended for a single discharge. In other implementations the power supply is a rechargeable battery, configured to discharge and recharge. The wireless communications components include at least one transceiver, and in some implementations, the wireless communications components include means to program the transceiver.

FIGS. 1A and 1B show a wireless patient monitoring pad 105, a power supply 130, a pull tab 135 that inserts into the power supply 130, and a user 115. The power supply 130 provides power to circuitry inside the patient monitoring pad 105. The circuitry inside the wireless patient monitoring pad 105 is circuitry for detecting the status of a patient and circuitry for wirelessly transmitting, such as sending or receiving, information about the patient status or programming of the circuitry within the wireless patient monitoring pad 105. The pull tab 135 prevents power from flowing from the power supply 130 to the circuitry inside the patient monitoring pad 105. The circuitry for detecting the status of a patient can be for example, a detector that detects whether a patient is sitting or lying on the pad.

FIG. 1C shows a wireless patient monitoring pad 105 with a pull tab 135 inserted in a position that prevents power from flowing from the power supply (130 in FIG. 1B). A user 115 is shown holding the pull tab 135. FIG. 1D shows the wireless patient monitoring pad 105 with the pull tab 135 removed by the user 115. With the pull tab 135 removed, power flows from the power supply 130 to the circuitry within the wireless patient monitoring pad 105. An indicator light 140 lights once power flows through the circuitry, after the user 115 removes the pull tab 135 from the wireless patient monitoring pad 105. The indicator light 140 provides an indication as to whether or not the pull tab is inserted into the power supply. For example, the light may be on to indicate that the pull tab is indeed inserted into the power supply, or may be on (or a different color than a previous state) to indicate the pull tab removed from the power supply.

The circuitry within the self-contained, wireless patient monitoring pad includes circuitry to monitor the status of a patient and signal transmitting components. The transmitting components include at least one transceiver, and can include memory components, as well. The self-contained, wireless patient monitoring pad can include programming circuitry that determines the monitoring activity of the self-contained, wireless patient monitoring pad. For example, the self-contained, wireless patient monitoring pad can have a finite lifetime, and the programming circuitry can signal or determine the start of the finite lifetime of the monitoring pad. The programming circuitry can detect the onset of use of the self-contained, wireless patient monitoring pad and calculate an end of the lifetime for the monitoring pad and, in some embodiments, activate the power supply. The programming circuitry can also interpret monitoring signals according to rules and trigger alarms accordingly. The rules can be pre-programmed and activated by switches, or the rules can be programmed via connection of the self-contained, wireless patient monitoring pad with an external computing device, either wirelessly or through a wired connection, such as through a USB or other communication port.

The programming circuitry, transmitting circuitry, and power supplying circuitry, including a power supply, can be built-in the self-contained, wireless patient monitoring pad during manufacturing of the patient monitoring pad. The programming circuitry, power supplying circuitry with the power supply, and transmitting circuitry are located away from the bulk of the patient monitoring circuitry, particularly the portion of the patient monitoring pad on which a patient sits or lies while being monitored. By manufacturing the self-contained, wireless patient monitoring pad with built-in transmitting circuitry and a built in power supply, the patient monitoring pad can be straight forward to use, with no cables or lines to complicate positioning the self-contained, wireless patient monitoring pad. As mentioned above, the self-contained, wireless patient monitoring pad can have a finite lifetime, and in some embodiments, the patient monitoring pad can be disposable. In an embodiment, the patient monitoring pad has a padded region that is sized and shaped for a patient to sit upon, and the power supply and/or circuitry is not located in the padded region of the patient monitoring pad.

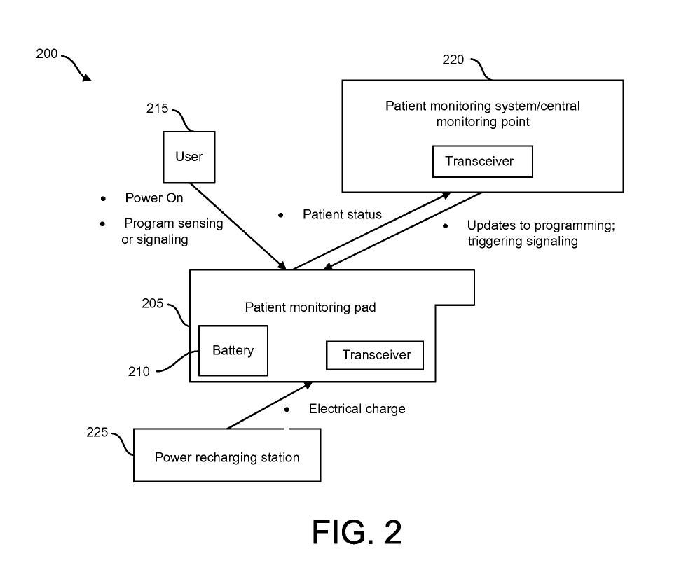

FIG. 2 shows a flow diagram 200 with exemplary flows of data between a patient monitoring system including a self-contained, wireless patient monitoring pad 205 and a monitoring point or base 220, as well as the interaction of a user 215 with the self-contained, wireless patient monitoring pad 205. The wireless patient monitoring pad 205 includes a battery 210 and a transceiver. The battery 210 and transceiver are built into the wireless patient monitoring pad 205 during manufacturing.

The user 215 interacts with the wireless patient monitoring pad 205 to power on the wireless patient monitoring pad 205 and to send programming information, such as programming regarding sensing or signaling, to the wireless patient monitoring pad 205. The user 215 powers on the wireless patient monitoring pad 205 by allowing power to flow from the battery 210 to circuitry within the wireless patient monitoring pad 205. The user 215 can power on the wireless patient monitoring pad 205 by removing a pull tab or other impediment that physically separates the battery 210 from the circuitry in the wireless patient monitoring pad 205. The user 215 can also use a flip or toggle switch, a magnetic switch, a software signal, or a combination thereof, as well as any of those along with a pull tab to power on the wireless patient monitoring pad 205. A power recharging station 225 can recharge the battery 210 in the self-contained, wireless patient monitoring pad 205, such as through induction charging or direct charging using a wired connection.

The user 215 can send programming information, such as information about the type of sensing or signaling to use, to the wireless patient monitoring pad 205. The user 215 can send this programming information to the wireless patient monitoring pad 205 through a flip or toggle switch, a magnetic switch, or a combination thereof. In some instances, the wireless patient monitoring pad 205 has more than one switch, either all of the same type or of more than one type of switch.

The patient monitoring base 220 has one or more transceivers which send and receive information from one or more transceivers in the wireless patient monitoring pad 205. The patient monitoring base 220 can have a central monitoring point (CMP) that can aggregate signals from multiple monitoring bases, such as multiple patient monitoring pads, and other devices in the patient monitoring system. Transceivers can be located in the central monitoring point or in another part of the patient monitoring base, such as other devices in the patient monitoring system. The transceivers of the patient monitoring base can be located in any of a bed monitor, a bed-side monitor, a central monitoring point, a pager, a hand-held device, a mobile phone, a Nursecall system, or a combination thereof. The transceivers of the self-contained, wireless patient monitoring pad can communicate directly with any of the transceivers of the patient monitoring base.

In some embodiments, the central monitoring point (CMP) of the patient monitoring base sends out queries at predetermined, regular intervals to the self-contained, wireless patient monitoring pad. The CMP transceiver sends the queries and receives any responses. The self-contained, wireless patient monitoring pad transceivers send and receive the queries. If the self-contained, wireless patient monitoring pad is operating correctly, then the patient monitoring pad can retrieve information from its memory component. The information can be the self-contained, wireless patient monitoring pad's current status or history information, including status history. The transceiver of the self-contained, wireless patient monitoring pad sends a response back to the transceiver of the central monitoring point or another part of the patient monitoring base. Each response can be stored in a memory component of the patient monitoring base. The CMP and the self-contained, wireless patient monitoring pad can be located in the same room or in different rooms. The CMP can be located in a nurses' station or near a doorway or other entrance to a ward.

Based upon the responses from the self-contained, wireless patient monitoring pad, or lack thereof, the patient monitoring base determines whether an alarm condition exists. An alarm condition can include lack of response from the self-contained, wireless patient monitoring pad within a predetermined amount of time and after sending a query at least 3 times. An alarm condition can also include an indication from the self-contained, wireless patient monitoring pad that a patient has moved, such as out of bed or away from a specific area. An alarm condition can require that the self-contained, wireless patient monitoring pad indicates that a patient has moved or changed condition. Once an alarm condition has been determined, the alarm system activates. The alarm system can activate an alarm signal, such as messages, visual indicators, audio indicators, or any combination thereof. The alarm system can provide an alarm signal and other indicators of the alarm condition at any part of the patient monitoring base, such as the central monitoring point, or to a remote location.

A care giver or user receives alarms from the central monitoring point of the patient monitoring base or directly from the self-contained, wireless patient monitoring pad when appropriate. The alarm can be received on any of a pager, a hand-held device, a mobile phone, or via a text-based message, such as a text short message, an e-mail, and the like. Additionally, the care giver or user can provide feedback or instructions to the patient monitoring base via the central monitoring point. Such feedback can control the central monitoring point or the self-contained, wireless patient monitoring pad, or the feedback may be used as instructions for other care givers, such as instructions from a doctor to a nurse in response to the alarm. Alternatively, in response to an alarm, a care giver or user can interact directly with the self-contained, wireless patient monitoring pad to ensure the health and safety of a patient and rectify the alarm condition.

The alarm signal can be an audio signal, a visual signal, or both an audio and visual signal. An audio signal can be a sustained sound, an instantaneous sound, and/or a repeating sound. A visual signal can be a flashing light, an indicator light, a message on a user interface, or any combination thereof. A message can be a message on a user interface, a message sent to an external device, or an audio message. A message can also be an audio telephonic message sent to a land-line, a mobile phone, or a voice-mail account. Additionally, a message can be a text-based or icon based message, such as a short message service message (i.e. SMS text message), an e-mail, or a multimedia messaging service message received on a mobile phone, pager, or hand-held device that is configured to send and receive data using cellular phone signaling means.

The self-contained, wireless patient monitoring pad can be used in any situation where monitoring of a patient or individual is desired. For example, the self-contained, wireless patient monitoring pad can be communicatively coupled to a Personal Emergency Alarm System (PERS) for on-site assisted living accommodation, to a nurses' station or off-site alarm relay via the telephone network or internet, to alert family, friends, caregivers, control centers, or any combination thereof. The patient monitoring system, particularly the central monitoring point, can also be communicatively coupled with Nursecall Systems in hospitals, nursing homes, and other assisted living facilities. Wireless connections between the transceivers in the self-contained, wireless patient monitoring pad and portions of a patient monitoring system, such as a caregiver's pager, a hand-held device, a mobile phone, a bed monitor, a bed-side patient monitor, or a the central monitoring point of the patient monitor system, can utilize any suitable wireless system, such as Bluetooth, WiFi, radio frequency, Zigbee communication protocols, infrared, cellular phone systems, and the like, and can also employ coding or authentication to verify the origin of the information received by any of the self-contained, wireless patient monitoring pad, a central point of the patient monitoring system, a pager, a hand-held device, a mobile phone, a bed monitor, a bed-side patient monitor, or any combination thereof.

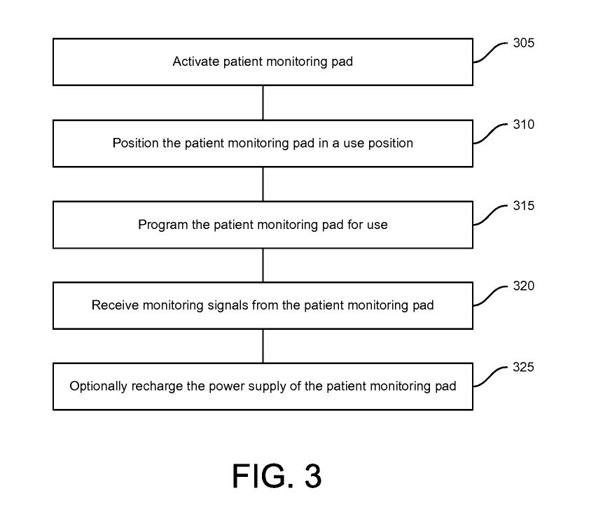

FIG. 3 shows an exemplary method using a self-contained, wireless patient monitoring pad. When a user receives a self-contained, wireless patient monitoring pad, the user activates the patient monitoring pad, as in box 305. The user activates the self-contained, wireless patient monitoring pad by activating the built-in power supply, such that the power supply provides power to the monitoring pad. The power supply provides power, such as electrical current, to the self-contained, wireless patient monitoring pad after the user allows power to transfer by removing a pull tab that isolates the power supply from circuitry in the self-contained, wireless patient monitoring pad or by flipping a switch or toggle. The user can also activate the power supply in the self-contained, wireless patient monitoring pad by using a magnetic switch, such that electrical current can be induced to flow using a magnet to create a current or move a physical element.

Once the user activates the self-contained, wireless patient monitoring pad, the user positions the patient monitoring pad into a use position, 310. A use position can include a position in a patient bed or chair, such as a wheel chair or chair in a care facility. The user can program the self-contained, wireless patient monitoring pad before or after positioning the patient monitoring pad into the use position. The user can use a switch, a user interface, a magnetic switch, a wireless communications means, or a combination thereof to program the self-contained, wireless patient monitoring pad, 315. The patient monitoring base receives monitoring signals from the self-contained, wireless patient monitoring pad, 320. The signals can be alarms, status signals, or the like, as described in greater detail hereinabove. The user can optionally recharge the power supply within the self-contained, wireless patient monitoring pad, 325. The user can recharge the power supply by wireless means, such as an inductive charging system or by using an electrical cable that plugs into a port in the self-contained, wireless patient monitoring pad. Alternatively, the self-contained, wireless patient monitoring pad can have built-in means for providing power to the built-in power supply, such as a dynamo, photovoltaic cells, or a combination thereof.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Although embodiments of various methods and devices are described herein in detail with reference to certain versions, it should be appreciated that other versions, methods of use, embodiments, and combinations thereof are also possible. Therefore the spirit and scope of the appended claims should not be limited to the description of the embodiments contained herein.

What is claimed is:

1. A wireless patient monitoring system, comprising:
a patient monitoring pad defining an interior cavity, the interior cavity containing a power supply, a pull tab inserted into the power supply, circuitry that detects a status of a patient, and a first transceiver configured to transmit a detected status of a patient, wherein the pull tab when inserted into the power supply blocks transfer of power from the power supply to at least one of the circuitry and the first transceiver;
a patient monitoring base adapted to wirelessly communicate with the patient monitoring pad, the patient monitoring base including a second transceiver adapted to receive a wireless communication signal from the first transceiver.

2. A system as in claim 1, wherein the power supply is a battery.

3. A system as in claim 2, wherein the power supply is a rechargeable battery.

4. A system as in claim 1, wherein circuitry detects whether a patient is on top of the pad.

5. A system as in claim 1, wherein circuitry detects whether a patient is near the pad.

6. A system as in claim 1, wherein the transceiver is programmable.

7. A system as in claim 1, wherein the pull tab is removable from the power supply, and wherein power flows from the battery to at least one of the circuitry and the first transceiver when the pull tab is removed from the power supply.

8. A system as in claim 7, further comprising an indicator light on the patient monitoring pad, the indicator light providing an indication as to whether the pull tab is inserted into the power supply.

9. A system as in claim 1, further comprising an indicator on the patient monitoring base to indicate the status of the patient.

10. A system as in claim 1, wherein the patient monitoring pad has a padded region that is sized and shaped for a patient to sit upon, and wherein the power supply is not located in the padded region of the patient monitoring pad.

11. A system as in claim 1, wherein at least one of the patient monitoring pad and the patient monitoring base emits an alarm signal related to the status of the patient.
